(19) United States
(12) Patent Application Publication
(10) Pub. No.: US 2026/0174685 A1
(43) Pub. Date: Jun. 25, 2026

(54) ORAL DISINTEGRATING FILM COMPRISING CANNABINOID AS ACTIVE INGREDIENT AND METHOD OF PREPARING THE SAME

(71) Applicant: BIO360 CO., LTD., Cheonan-si (KR)

(72) Inventors: Byoung Hag KIM, Cheonan-si (KR); Esther LYUH, Seoul (KR); Kun Se KIM, Seoul (KR); Yoo Jung KIM, Daegu (KR); Jeong Han MOON, Gwangju (KR)

(21) Appl. No.: 19/085,133

(22) Filed: Mar. 20, 2025

(30) Foreign Application Priority Data
Dec. 19, 2024 (KR) 10-2024-0191757

(51) Int. Cl. A61K 9/00; A61K 31/00; A61K 47/14; A61K 47/36

(52) U.S. Cl. CPC A61K 9/006; A61K 31/658; A61K 47/14; A61K 47/36

(57) ABSTRACT

Provided are an orally disintegrating film preparation containing cannabinoids, wherein the orally disintegrating film preparation according to an aspect enables the manufacture of a cannabinoid-containing oral disintegrating film with excellent physical properties, thereby improving the dosing convenience and bioavailability of cannabinoids.

### CROSS-REFERENCE TO RELATED APPLICATION

This application claims priority under 35 U.S.C. § 119 to Korean Patent Application No. 10-2024-0191757, filed on Dec. 19, 2024, the disclosure of which is incorporated by reference herein in its entirety.

### BACKGROUND

### 1. Field

Disclosed are a novel oral disintegrating film and a method of preparing the same.

### 2. Description of the Related Art

Cannabinoids are a group of diverse compounds that activate cannabinoid receptors, and include endocannabinoids, which are naturally synthesized in humans and animals, and phytocannabinoids, which are extracted from Cannabis plants. Major cannabinoids include tetrahydrocannabinol (THC), cannabidiol (CBD), and cannabinol (CBN). THC is particularly well-known as the main psychoactive component of marijuana. Cannabinoids exhibit anti-inflammatory, analgesic, and anxiolytic effects and are expected to find various applications, such as alleviating nausea, muscle stiffness, and pain caused by chemotherapy. However, considerations regarding side effects, safety, and legal regulations present difficulties in developing cannabinoid-based products.

Among existing dosage forms in which active ingredients must be disintegrated prior to ingestion, oral disintegrating film (ODF) formulations are regarded as an improved dosage form. Since ODFs can be taken without water, they are especially useful for the elderly, children, disabled individuals, and patients who have difficulty swallowing tablets or capsules. In addition to health-vulnerable populations, ODFs are garnering attention from busy modern consumers as a form of health supplement due to their advantages: convenience of carrying and administration, and rapid absorption of active ingredients.

Accordingly, based on the ingestion of cannabinoids and its resulting enhancement of various physiological activities, the inventors initiated research on ODF formulations. ODFs face several disadvantages: they cannot be produced beyond a certain weight, as they must be thin enough to ensure rapid disintegration in the oral cavity; they can only accommodate a relatively low amount of active ingredients, since a certain quantity of polymer must be included to ensure proper blending with the drug and to maintain strength and plasticity suitable for handling of the film; and their manufacturing method varies significantly depending on the form and amount of the raw material that provides the primary pharmacological effect, making standardization difficult. Therefore, developing an orally disintegrating formulation tailored to the raw material characteristics of the main active ingredient is crucial.

With these considerations in mind, the inventors endeavored to determine the optimal formulation of an ODF containing cannabinoids and identified the optimal amounts of various additives, thereby completing the present invention.

### SUMMARY

One aspect is to provide an oral disintegrating film preparation including cannabinoids and medium-chain-triglyceride oil (MCT) oil.

Another aspect is to provide a method of preparing oral disintegrating film.

One aspect provides an oral disintegrating film preparation including cannabinoids and MCT oil.

In the present specification, the term “oral disintegrating film (ODF)” is defined in accordance with the product form definitions outlined in the Health Functional Food Code and is also referred to as a “strip”, “orally dissolving film”, or “edible film”. Specifically, the ODF refers to a preparation that is absorbed into capillaries through the mucosa in the oral cavity, and a film formulation that can be dissolved or finely dispersed in the oral cavity for ingestion. This film formulation may dissolve on the tongue, but may also be adhered to the palate, sublingual area, buccal cavity, oral mucosa, inner side of the cheek, or supragingival area for ingestion. Preferably, the formulation may dissolve, disperse, or disintegrate 80% or more of the entire preparation within 1 minute or less after oral administration.

In the present specification, the term “oral disintegrating film preparation” refers to a mixture containing the components necessary for preparing the oral disintegrating film of the present disclosure, and includes, without limitation, mixture forms prior to the drying process for preparing the oral disintegrating film.

In the present specification, the term “cannabinoid” refers to chemical compounds found in cannabis (hemp plants).

The aforementioned cannabinoid may be cannabidiol (CBD). CBD is a non-psychoactive component with various medical effects, including sedation, anti-inflammatory, and pain relief. Unlike THC, CBD does not induce hallucinogenic effects and can be researched and used for the treatment of various diseases or as a health supplement.

The cannabidiol may be a cannabidiol extract. In the present specification, the term “extract” includes, without limitation, an extract solution obtained through the extraction processing of the target substance, a diluted or concentrated solution of the extract solution, a dried product such as a solid component obtained by drying the extract solution, an adjusted product or purified product of the extract solution, or a mixture thereof, as well as the extract itself and all extract formulations that can be formed using the extract. In the present specification, cannabidiol may be in the form of pure cannabidiol (CBD) isolate. In other words, the cannabidiol may be a “CBD isolate” or “CBD pure extract” in which all other compounds extracted from cannabis or hemp (e.g., THC, terpenes, flavonoids, etc.) are removed and only CBD remains, or it may be a powder obtained by drying and grinding thereof.

In an embodiment, the cannabinoids may be included in an amount of about 1 wt % to about 20 wt %, about 1 wt % to about 10 wt %, about 1 wt % to about 8 wt %, about 1 wt % to about 6 wt %, about 3 wt % to about 10 wt %, about 5 wt % to about 8 wt %, or about 5 wt % to about 6 wt %, relative to the total weight of the oral disintegrating film preparation of the present disclosure.

The oral disintegrating film preparation of the present disclosure may include a film-forming agent for preparing an oral disintegrating film.

In the present specification, the term “film-forming agent” or “film-forming polymer” refers to a water-soluble polymer that forms oral disintegrating films. The film-forming agent may include pullulan, gelatin, hydroxypropyl methylcellulose, modified starch, maltodextrin, and the like.

In an embodiment, the film-forming agent may be included in an amount of about 10 wt % to about 40 wt %, about 15 wt % to about 40 wt %, about 20 wt % to about 40 wt %, about 15 wt % to about 30 wt %, about 20 wt % to about 30 wt %, about 22 wt % to about 25 wt %, or 25 wt % or less, relative to the total weight of the oral disintegrating film preparation of the present disclosure.

In an embodiment, the film-forming agent may include maltodextrin.

The maltodextrin may be included in an amount of about 10 wt % to about 40 wt %, about 15 wt % to about 40 wt %, about 20 wt % to about 40 wt %, about 25 wt % to about 40 wt %, about 15 wt % to about 35 wt %, about 20 wt % to about 30 wt %, about 25 wt % to about 30 wt %, about 26 wt % to about 28 wt %, or 30 wt % or less, relative to the total weight of the oral disintegrating film preparation of the present disclosure.

In an embodiment, the film-forming agent may be a combination of maltodextrin and pullulan.

In an embodiment, the film-forming agent may be a combination of maltodextrin, pullulan, and hydroxypropyl methylcellulose.

In an embodiment, the film-forming agent may include maltodextrin in an amount of about 10 wt % to about 20 wt %, about 15 wt % to about 20 wt %, or about 17 wt % to about 18 wt %, relative to the total weight of the oral disintegrating film preparation of the present disclosure; and
pullulan in an amount of about 5 wt % to about 15 wt %, about 7 wt % to about 12 wt %, or about 9 wt % to about 10 wt % relative to the total weight of the oral disintegrating film preparation of the present disclosure.

In an embodiment, the maltodextrin and pullulan may be included in a weight ratio of about 0.1 to 10:1, about 0.5 to 5:1, about 0.6 to 2:1, about 1:2, about 1:1, or about 2:1.

Meanwhile, cannabinoids due to their low water solubility, require an emulsification process during the film-forming solution preparation. However, selecting an emulsifier that is not suitable for the active ingredient or adding an excessive amount of emulsifier may result in poor film formation. Additionally, high concentrations of cannabinoids may cause grassy or bitter tastes, necessitating taste masking; however, excessive inclusion of additives may increase compositional complexity, potentially affecting the film's dissolution and absorption rates, and may leave a residual sensation in the mouth. Furthermore, since cannabinoids are susceptible to oxidation and light-sensitive, making them vulnerable to oxidative degradation, antioxidants must be incorporated; however, excessive inclusion of antioxidants may cause alterations in taste and odor, irritate the oral mucosa, affect the film's dissolution and absorption rates, and undergo interactions with other ingredients, leading to product degradation during storage. Therefore, when preparing cannabinoid films, these factors must be taken into account when combining excipients and additives in order to produce an edible form.

An oral disintegrating film prepared using the preparation of the present disclosure may effectively solubilize and disperse cannabinoids in the aqueous phase by including an appropriate proportion of emulsifier, preferably, MCT oil.

In an embodiment, the emulsifier may include MCT oil.

In an embodiment, the MCT oil may be included in an amount of about 0.1 wt % to about 2 wt %, about 0.1 wt % to about 1.5 wt %, about 0.1 wt % to about 1 wt %, about 0.1 wt % to about 0.7 wt %, about 0.1 wt % to about 0.5 wt %, about 0.3 wt % to about 1 wt %, about 0.3 wt % to about 0.7 wt %, or about 0.3 wt % to about 0.5 wt %, relative to the total weight of the oral disintegrating film preparation of the present disclosure.

In an embodiment, the MCT oil may be included in an amount of less than 1 wt %, 0.7 wt % or less, 0.5 wt % or less, or 0.4 wt % or less, relative to the total weight of the oral disintegrating film preparation of the present disclosure.

In an embodiment, the emulsifier may include a combination of MCT oil and glycerin.

In an embodiment, the glycerin may be included in an amount of about 0.1 wt % to about 1 wt %, about 0.1 wt % to about 0.5 wt %, about 0.1 wt % to about 0.2 wt %, about 0.15 wt % to about 0.25 wt %, or 0.2 wt % or less, relative to the total weight of the oral disintegrating film preparation of the present disclosure.

In an embodiment, the MCT oil may be included in an amount of 0.4 wt % or less relative to the total weight of the oral disintegrating film preparation of the present disclosure; and the glycerin may be included in an amount of 0.2 wt % or less relative to the total weight of the oral disintegrating film preparation of the present disclosure.

In an embodiment, the MCT oil and the glycerin may be included in a weight ratio of about 1:0.1 to 1, about 1:0.5 to 1, or about 1:0.6 to 0.7.

In an embodiment, the MCT oil and the glycerin may be included in a weight ratio of about 2:1.

The oral disintegrating film preparation of the present disclosure may include appropriate excipients and/or additives in amounts typically used as needed.

The excipients are substances added to improve disintegration, solubility, molding properties, tabletability, stability, flexibility, elasticity, etc., and may include, for example, plasticizers, chelating agents, disintegrants, stabilizers, colorants, dyes, etc., but are not limited thereto and encompass all forms suitable for use in oral disintegrating film preparations.

The additives may include vitamins, nutritional enhancers, acidity regulators, minerals, sweeteners, souring agents, flavoring agents, flavor enhancers, antifoaming agents, etc., but are not limited thereto and encompass all forms suitable for use in oral disintegrating film preparations. These additives are incorporated for purposes such as forming, stabilization, taste improvement, and nutritional enhancement, and may be incorporated in amounts that do not adversely affect the disintegration time or hardness of the oral disintegrating film preparation.

The excipients and additives may be added individually or in combination.

In an embodiment, the plasticizer may include acetyl triethyl citrate, polyethylene glycol, propylene glycol, polypropylene glycol, glycerin, citrate ester, triacetin, triethyl citrate, or polysorbate 80. The plasticizer may be included in an amount of about 0.1 wt % to about 5 wt %, about 0.1 wt % to about 4 wt %, about 0.1 wt % to about 3 wt %, about 0.1 wt % to about 2 wt %, about 0.5 wt % to about 5 wt %, about 0.5 wt % to about 3 wt %, about 0.5 wt % to about 2 wt %, about 1 wt % to about 5 wt %, about 1 wt % to about 4 wt %, about 1 wt % to about 3 wt %, or about 1 wt % to about 2 wt %, relative to the total weight of the solids of the oral disintegrating film preparation of the present disclosure.

In an embodiment, the disintegrant may include, without limitation, disintegrants known in the art, such as sodium carboxymethyl cellulose (CMC-Na), calcium carboxymethyl cellulose (CMC-Ca), or modified starch. The modified starch may be an acid-treated modified starch, hydroxypropylated modified starch, α-modified starch, cross-linked modified starch, and modified starches made from starch hydrolysates and has the advantage of favorable film-forming ability comparable to that of pullulan. The disintegrant may be included in an amount of about 0.1 wt % to about 5 wt %, for example, about 0.1 wt % to about 5 wt %, about 0.1 wt % to about 3 wt %, about 0.1 wt % to about 1 wt %, about 0.3 wt % to about 1 wt %, about 0.5 wt % to about 1 wt %, about 0.7 wt % to about 1 wt %, or about 0.9 wt % to about 1 wt %, relative to the total weight of the solids of the oral disintegrating film preparation of the present disclosure.

In an embodiment, the nutritional enhancer may include, without limitation, nutritional enhancers known in the art, such as vitamins, free amino acids such as leucine, isoleucine, valine, or glutamine, and other compounds such as carnitine, taurine, taurine salts, or arginine.

In an embodiment, the vitamins may include, without limitation, vitamins known in the art, such as vitamin A or its esters (e.g., vitamin A acetate and vitamin A palmitate), vitamin E or its esters (e.g., vitamin E acetate), vitamin K (phytomenadione), vitamin D3 (cholecalciferol), vitamin B1 (thiamine), vitamin B2 (riboflavin), vitamin B3 (niacin, niacinamide), vitamin B5 (pantothenic acid), vitamin B6 (pyridoxine, pyridoxamine, pyridoxal), vitamin B7 (biotin), vitamin B9 (folic acid, folinic acid), vitamin B12 (cyanocobalamin, hydroxocobalamin, methylcobalamin), or vitamin C (ascorbic acid).

In an embodiment, the minerals may include, without limitation, minerals known in the art, such as iron, zinc, calcium, selenium, copper, boron, iodine, manganese, chromium, molybdenum, arsenic, or cobalt.

In an embodiment, the souring agent may include, without limitation, souring agents known in the art, such as citric acid, malic acid, or tartaric acid.

In an embodiment, the sweetener may include, without limitation, sweeteners known in the art, such as sugars and sugar alcohols, including agave powder, xylitol, erythritol, sorbitol, trehalose, glucose, and sucrose, as well as high-intensity sweeteners, including aspartame, sucralose, saccharin and its salts, dipotassium glycyrrhizinate, thaumatin, and stevia.

In an embodiment, the flavor enhancer may include, without limitation, flavor enhancers known in the art, such as natural flavoring agents (e.g., guarana, orange, lemon, peppermint, cinnamon, menthol, peppermint oil, wintergreen mint, clove, butterscotch, maple, apricot, peach flavor, cherry flavor, anise, strawberry flavor, vanillin, citrus flavor, pineapple, licorice flavor, root beer flavor, gardenia, raspberry flavor, grape flavor, tropical flavor, walnut flavor, chocolate flavor, etc.) and/or synthetic flavoring agents.

Another aspect provides a method of preparing an orally disintegrating film, the method including: heating and dissolving a mixture of cannabinoids and a solvent, followed by cooling;

preparing a film-forming solution by adding MCT oil and a film-forming agent to the mixture and stirring; and

drying the film-forming solution.

In an embodiment, the preparing of the film-forming solution may further include adding an excipient or additive, followed by stirring. The excipient or additive is as described above.

In an embodiment, the drying of the film-forming solution may include applying the film-forming solution onto a plate and drying. Specifically, the film-forming solution may be applied to a thickness of about 500 μm to about 1,500 μm, about 600 μm to about 1,300 μm, or about 700 μm to about 1,000 μm.

In an embodiment, the drying may be carried out for about 10 minutes to about 60 minutes, about 15 minutes to about 45 minutes, about 20 minutes to about 40 minutes, or about 20 minutes to about 35 minutes.

In an embodiment, the drying may be carried out at a temperature of about 50° C. to about 100° C., about 60° C. to about 90° C., or about 75° C. to about 85° C.

Since the present disclosure allows for various modifications and may have numerous embodiments, particular embodiments will be illustrated in the drawings and described in detail in the detailed description. However, this is not intended to limit the present disclosure to particular modes of practice, and it is to be appreciated that all modifications, equivalents, and substitutes that do not depart from the spirit and technical scope of the present disclosure are encompassed in the present disclosure. In describing the present disclosure, detailed descriptions of known art that might obscure the gist of the present disclosure are omitted herein.

### DETAILED DESCRIPTION

The following examples are provided to illustrate the present disclosure in further detail. However, these examples are for illustrative purposes only and should not be construed as limiting the scope of the present disclosure. Throughout the present specification, when one element is described as “comprising,” and/or “including” another element, this does not preclude the presence or addition of one or more other elements, unless otherwise indicated.

### EXAMPLES

### (1) Preparation Process for the CBD Film

Oral disintegrating films containing the raw materials and composition shown in the table below were prepared according to the following process. Table 1 lists the ingredients used in film preparation and their basic mixing ratios.

TABLE 1

| Phase | Component | Amount (wt %) |
|---|---|---|
| A | Active Ingredient (CBD isolate) | 3-10 |
| | Emulsifier | 0.1-1 |
| | Stabilizer | 1-3 |
| B | Nutritional Enhancer | 0.1-1 |
| | Acidity Regulator | 1.2-2.5 |
| | Colorant | 1-2 |
| | Sweetener | 1-1.5 |
| | Flavoring Agent | 4.5-6 |
| C | Film-Forming Agent | 20-30 |
| Processing | Purified Water | To 100 |
| Total | | 100 |

First, purified water, the active ingredients of Phase A, an emulsifier, and a stabilizer were added and dispersed at room temperature for 30 minutes with stirring. Subsequently, the raw materials of Phase B (nutritional enhancer, acidity regulator, colorant, sweetener, and flavoring agent) were sequentially added, and stirring continued at room temperature for 30 minutes. Thereafter, the raw materials of Phase C were added, followed by stirring at room temperature for 60 minutes. Next, the resulting mixture was degassed at 2,000 RPM for 5 minutes to prepare the forming solution. The forming solution was then applied to a base film plate at a thickness of 800 μm to 1,000 μm, dried at 80° C. for 20 minutes to 35 minutes, and subsequently cut to dimensions of 25 mm×40 mm to yield the final oral disintegrating film.

### (2) Selection of Candidate Emulsifiers

Due to the inherent properties of CBD, a suitable emulsifier is required to emulsify CBD in the forming solution. Candidate emulsifiers considered suitable for developing the CBD film of the present disclosure included lecithin, glycerin, Tween 20, Tween 80, polyglyceryl fatty acid esters, sucrose fatty acid esters, polyethylene glycol 400, polysorbate 80, polysorbate 20, and MCT oil.

To select the most suitable emulsifier among these candidate emulsifiers for preparing the CBD film-forming solution, CBD isolate powder and each candidate emulsifier were mixed at a ratio of 5:1. Water was then added, and the degree of dissolution was visually assessed and evaluated according to the following criteria: The evaluation criteria were as follows.

1 point: Agglomerated and undissolved.

2 points: Partially dissolved with some agglomeration, resulting in a cloudy appearance.

3 points: Finely dispersed with slight turbid appearance.

4 points: Uniformly solubilized.

TABLE 2

| | | Emulsification |
|---|---|---|
| No. | Candidate emulsifier | score |
| 1 | Lecithin | 2 |
| 2 | Glycerin | 3 |
| 3 | Tween20 | 1 |
| 4 | Tween80 | 2 |
| 5 | Polyglycerin fatty acid ester | 3 |
| 6 | Sucrose fatty acid ester | 1 |
| 7 | Polyethylene glycol 400 | 2 |
| 8 | Polysorbate 80 | 4 |
| 9 | Polysorbate 20 | 3 |
| 10 | MCT oil | 4 |

As a result, as shown in Table 2, among the emulsifiers generally known in the art, there were components unsuitable for uniformly solubilizing CBD in an aqueous phase without crystallization or precipitation. Among the emulsifiers, polysorbate 80 and MCT oil, among others, were selected as suitable candidate emulsifiers for preparing the CBD film-forming solution.

### (3) Selection of Optimal Film-Forming Agent and Emulsifier

Even if an emulsifier is suitable for solubilizing CBD isolate, when mixed with various components in an oral disintegrating film, it may not form properly into a film. In addition, when preparing an oral disintegrating film, it is essential to select a film-forming agent appropriate for the main ingredient. To select the optimal film-forming agent and emulsifier for preparing a CBD oral disintegrating film and to determine their optimal weight ratios, the oral disintegrating films for Examples 1 to 11 and Comparative Examples 1 to 8 were prepared by mixing each component according to the compositions shown in Table 3.

TABLE 3

| | Amount (wt %) | | | | | | | | |
|---|---|---|---|---|---|---|---|---|---|
| | Main | | | | | | | | Processing |
| | Component | Emulsifier | | | Film-Forming Agent | | | | Solvent |
| | CBD | Polysorbate | MCT | | | | Modified | | Purified |
| Item | Powder | 80 | Oil | Glycerin | Pullulan | HPMC | Starch | Maltodextrin | Water |
| Example 1 | 5.5 | — | 0.5 | — | — | — | — | 24 | To |
| | | | | | | | | | 100 |
| Example 2 | 5.5 | — | 0.5 | — | 8 | — | — | 16 | To |
| | | | | | | | | | 100 |
| Example 3 | 5.5 | — | 0.5 | — | 16 | — | — | 8 | To |
| | | | | | | | | | 100 |
| Example 4 | 5.5 | — | 0.5 | — | 16 | 1 | — | 7 | To |
| | | | | | | | | | 100 |
| Example 5 | 5.5 | — | 0.7 | — | 8 | — | — | 16 | To |
| | | | | | | | | | 100 |
| Example 6 | 5.5 | — | 0.3 | — | 8 | — | — | 16 | To |
| | | | | | | | | | 100 |
| Example 7 | 5.5 | — | 0.1 | — | 8 | — | — | 16 | To |
| | | | | | | | | | 100 |
| Example 8 | 5.5 | — | 0.3 | 0.2 | — | — | — | 24 | To |
| | | | | | | | | | 100 |
| Example 9 | 5.5 | — | 0.3 | 0.2 | 8 | — | — | 16 | To |
| | | | | | | | | | 100 |
| Example 10 | 5.5 | — | 0.3 | 0.2 | 16 | — | — | 8 | To |
| | | | | | | | | | 100 |
| Example 11 | 5.5 | — | 0.3 | 0.2 | 16 | 1 | — | 7 | To |
| | | | | | | | | | 100 |
| Comparative | 5.5 | — | 2 | — | 8 | — | — | 16 | To |
| Example 1 | | | | | | | | | 100 |
| Comparative | 5.5 | — | 1 | — | 8 | — | — | 16 | To |
| Example 2 | | | | | | | | | 100 |
| Comparative | 5.5 | — | 0.5 | — | 24 | — | — | — | To |
| Example 3 | | | | | | | | | 100 |
| Comparative | 5.5 | — | 0.5 | — | 16 | 1 | 7 | — | To |
| Example 4 | | | | | | | | | 100 |
| Comparative | 5.5 | 0.5 | — | — | 24 | — | — | — | To |
| Example 5 | | | | | | | | | 100 |
| Comparative | 5.5 | 0.5 | — | — | 16 | 1 | 7 | — | To |
| Example 6 | | | | | | | | | 100 |
| Comparative | 5.5 | 0.5 | — | — | 8 | — | — | 16 | To |
| Example 7 | | | | | | | | | 100 |
| Comparative | 5.5 | 0.3 | — | 0.2 | 8 | — | — | 16 | To |
| Example 8 | | | | | | | | | 100 |

### Experimental Example 1. Evaluation of Overall Film Quality

The oral disintegrating films prepared according to Examples and Comparative Examples and issues during their respective preparation processes were assessed.

As shown in Table 4 below, during the preparing of CBD films, it was observed that maltodextrin or combinations containing maltodextrin exhibited superior film peelability compared to pullulan, HPMC, modified starch, or combinations thereof. Specifically, because the CBD raw material contains a large amount of dietary fiber, it disrupted the molecular structure of pullulan, whereas this phenomenon was mitigated when mixed with maltodextrin. In addition, with respect to the emulsifier, using MCT oil alone or a combination of MCT oil and glycerin resulted in the best film peelability. The use of polysorbate 80 resulted in incomplete dissolution of raw materials, leading to either inability to form films or an excessively strong bitter taste.

TABLE 4

| Item | Issues with the formed film |
|---|---|
| Example 1 | Although the forming solution was somewhat diluted, the |
| | film formed well with favorable peelability. |
| Example 2 | None |
| Example 3 | None |
| Example 4 | None |
| Example 5 | None |
| Example 6 | None |
| Example 7 | Although emulsification took some time, no issues arose in |
| | film formation. |
| Example 8 | Although the forming solution was somewhat diluted, the |
| | film formed well with favorable peelability. |
| Example 9 | None |
| Example 10 | None |
| Example 11 | None |
| Comparative Example 1 | A layer of oil was observed in the forming solution, and fat |
| | was released on the film surface during film formation. |
| Comparative Example 2 | Fat was released on the film surface during film formation. |
| Comparative Example 3 | Because the CBD raw material contains large amounts of |
| | dietary fiber, it disrupted the molecular structure of pullulan, |
| | resulting in poor film formation. |
| Comparative Example 4 | Although the disruption of pullulan's molecular structure |
| | was somewhat alleviated, signs of disruption remained. |
| Comparative Example 5 | Film formation from the forming solution was not possible. |
| Comparative Example 6 | Film formation from the forming solution was not possible. |
| Comparative Example 7 | Fat was released on the film surface, and the bitterness |
| | was excessively strong. |
| Comparative Example 8 | Fat was released on the film surface, and the bitterness |
| | was excessively strong. |

### Experimental Example 2. Sensory and Physical Property Evaluation

A sensory test was conducted to evaluate preferences for the CBD films. In order to minimize sequence bias, the sample presentation order was randomized. The sensory panel consisted of 30 adult men and women between their 20s and 50s, all of whom had undergone appropriate training for sensory evaluation. The sensory evaluation form provided to the panel was the same as Table 5 below.

TABLE 5

| | | | | | | | | |
|---|---|---|---|---|---|---|---|---|
| | | | | Neither | | | | |
| Extremely | Strongly | | Somewhat | like nor | Somewhat | | Strongly | Extremely |
| dislike | dislike | Dislike | dislike | dislike | like | Like | like | like |

Sensory test

| | | | | | | | | | |
|---|---|---|---|---|---|---|---|---|---|
| <Overall taste | | | | | | | | | |
| evaluation> | | | | | | | | | |
| How do you | 1 | 2 | 3 | 4 | 5 | 6 | 7 | 8 | 9 |
| perceive | | | | | | | | | |
| the overall | | | | | | | | | |
| characteristics? | | | | | | | | | |

Please describe the reasons for your overall preference.
(e.g., reasons for likes and dislikes)

| | | | | | | | | | |
|---|---|---|---|---|---|---|---|---|---|
| <Sensory | | | | | | | | | |
| Evaluation> | | | | | | | | | |
| How favorable | 1 | 2 | 3 | 4 | 5 | 6 | 7 | 8 | 9 |
| is the film's | | | | | | | | | |
| taste? | | | | | | | | | |
| How favorable | 1 | 2 | 3 | 4 | 5 | 6 | 7 | 8 | 9 |
| is the film's | | | | | | | | | |
| aroma? | | | | | | | | | |
| How favorable | 1 | 2 | 3 | 4 | 5 | 6 | 7 | 8 | 9 |
| is the film's | | | | | | | | | |
| texture? | | | | | | | | | |

Please provide any additional comments. (e.g., unusual observations, areas for improvement, etc.)

As shown in Table 6, the overall preference ratings for Examples 1 to 11 were higher than those of Comparative Examples 1 to 8. In particular, Comparative Examples 5 and 6 (without MCT oil) and Example 7 (with relatively low MCT oil content) received predominantly negative feedback about strong bitterness despite acceptable texture, resulting in lower overall preference ratings. In Comparative Examples 1 and 2, which contained large amounts of MCT oil, evaluators noted that oil had seeped to the film's surface, causing a taste that was not harmonious. In the products using polysorbate 80, the taste was harmonious overall but exhibited a strong bitter note.

Additionally, in Comparative Examples 3 and 4, which did not contain maltodextrin, the film's texture was described as stiff, and evaluators reported an irritating sensation upon swallowing, thereby lowering the overall preference ratings.

TABLE 6

| | | | | Overall |
|---|---|---|---|---|
| Item | Taste | Flavor | Texture | Preference |
| Example 1 | 7.5 | 7.4 | 7.3 | 7.40 |
| Example 2 | 7.5 | 7.6 | 7.4 | 7.50 |
| Example 3 | 7.6 | 7.6 | 7.7 | 7.63 |
| Example 4 | 7.6 | 7.5 | 7.5 | 7.53 |
| Example 5 | 7.4 | 7.2 | 7.1 | 7.23 |
| Example 6 | 7.2 | 7.4 | 7.4 | 7.33 |
| Example 7 | 7.0 | 7.2 | 7.4 | 7.20 |
| Example 8 | 7.3 | 7.3 | 7.1 | 7.23 |
| Example 9 | 7.4 | 7.2 | 7.2 | 7.27 |
| Example 10 | 7.2 | 7.0 | 7.5 | 7.23 |
| Example 11 | 7.2 | 7.3 | 7.5 | 7.33 |
| Comparative | 6.6 | 5.9 | 7.0 | 6.50 |
| Example 1 | | | | |
| Comparative | 6.7 | 6.0 | 7.1 | 6.60 |
| Example 2 | | | | |
| Comparative | 6.9 | 6.8 | 6.4 | 6.70 |
| Example 3 | | | | |
| Comparative | 6.8 | 6.0 | 6.3 | 6.37 |
| Example 4 | | | | |
| Comparative | 5.8 | 5.6 | 6.8 | 6.07 |
| Example 7 | | | | |
| Comparative | 5.3 | 5.6 | 6.9 | 5.93 |
| Example 8 | | | | |

### Experimental Example 3. Measurement of the Final Physical Properties of the CBD Films

### 3.1 Determination of Optimal Composition Ratio

Based on the results of overall film quality evaluation and sensory evaluation tests, the composition shown in Table 7 below was selected as the final composition.

TABLE 7

| Functional | | Amount | |
|---|---|---|---|
| Category | Raw Material Name | (wt %) | Solids (%) |
| Active | CBD Powder | 5.50 | 12.24 |
| Ingredient | (98.51%; Biopharm) | | |
| Emulsifier | MCT Oil | 0.37 | 0.82 |
| Emulsifier | Glycerin | 0.18 | 0.41 |
| Stabilizer | CMC calcium | 0.55 | 1.22 |
| Stabilizer | Propylene Glycol | 0.92 | 2.04 |
| Stabilizer | β-CD | 0.92 | 2.04 |
| Nutritional | Vitamin E Complex | 0.55 | 1.22 |
| Acidity | Citric Acid | 1.65 | 3.67 |
| Colorant | Green Color Powder | 0.92 | 2.04 |
| Colorant | Yellow Green powder | 0.37 | 0.82 |
| Sweetener | Aspartame | 0.73 | 1.63 |
| Sweetener | Agave Powder | 0.37 | 0.82 |
| Flavoring | Orange Concentrate Powder | 3.12 | 6.94 |
| Flavoring | Lemon Fruit Powder | 0.37 | 0.82 |
| Natural | Natural Lemon Flavoring | 1.28 | 2.86 |
| Film- | Maltodextrin | 17.98 | 40.00 |
| Film- | Pullulan | 9.17 | 20.41 |
| Processing | Purified Water | To 100 | — |

| | |
|---|---|
| Total | 100 |

### 3.2 Evaluation of Antioxidant Activity (DPPH Free Radical Scavenging)

The antioxidant activity of the CBD oral disintegrating film prepared in Section 3.1 was evaluated. Specifically, the film was uniformly dissolved to 1%, then centrifuged at 10,000 rpm for 5 minutes at 4° C. to obtain a cell-free extract (CFE). 500 μL of the prepared CFE was mixed with 3.0 mL of a 2,2-diphenyl-2-picryl hydrazyl hydrate (DPPH) solution (5 mg/100 mL ethanol), and the same volume of ethanol, MRS solution, and ascorbic acid (100 μg/mL) were used as the control, blank, and positive control, respectively. After mixing with the DPPH solution and incubating in the dark for 30 minutes, the absorbance at 517 nm was measured and the antioxidant activity was expressed as % using the following equation:

$$DPPH - Free Radical Scavenging ( \% ) = ( ( Absorbance of Control - Absorbance of Sample ) / Absorbance of Control ) * 100 \qquad Equation 1$$

As a result, the DPPH radical scavenging activity of the CBD oral disintegrating film was found to be 81%, indicating excellent antioxidant capacity. These findings confirm that the CBD film described herein exhibits antioxidant activity.

### 3.3 Evaluation of Storage Stability

The CBD oral disintegrating film prepared in Section 3.1 was stored for three months under accelerated conditions (45° C., 75% RH), and changes in weight, moisture content, and peelability were observed at three-week intervals. The results are shown in Table 8. Surface moisture was measured using a surface moisture meter (HK-300, KET, Korea). Peelability was assessed by observing whether the film could be peeled off in a single motion without wrinkling, stickiness, or tearing. If the film peeled off cleanly, it was rated as “O”; if it did not peel easily with minimal force and showed slight warping or stickiness, it was marked “Δ”; and if the film warped, became sticky, or tore and could not be peeled properly, it was marked “X”.

TABLE 8

| Time | | | | Peelability |
|---|---|---|---|---|
| (week) | Weight (mg) | Surface Moisture | pH | Evaluation |
| 0 | 235.10 ± 7.01 | 5.10 ± 0.44 | 6.02 ± 0.20 | ◯ |
| 3 | 234.44 ± 9.87 | 5.07 ± 0.36 | 6.00 ± 0.16 | ◯ |
| 6 | 230.05 ± 9.31 | 4.99 ± 0.17 | 5.85 ± 0.11 | ◯ |
| 9 | 229.33 ± 10.11 | 5.00 ± 0.22 | 5.95 ± 0.09 | ◯ |
| 12 | 226.10 ± 0.11 | 4.91 ± 0.43 | 5.75 ± 0.01 | ◯ |

(Mean ± SD, n = 10)

As shown in Table 8, the oral disintegrating film stored for three months exhibited stable peelability without significant changes in weight, moisture, or pH.

These results indicate that, when MCT oil is selected as the emulsifier to prepare the film forming solution and manufacture the CBD-containing oral disintegrating film, it achieves excellent peelability, favorable sensory attributes, and storage stability, thereby enhancing the convenience of ingestion. In particular, using maltodextrin—or a combination of maltodextrin and pullulan—as the film-forming agent may yield a CBD film with excellent physical properties.

An oral disintegrating film preparation according to one aspect enables the manufacture of a cannabinoid-containing oral disintegrating film with excellent physical properties, thereby improving the dosing convenience and bioavailability of cannabinoids.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope as defined by the following claims.

What is claimed is:

1. An oral disintegrating film preparation comprising: cannabinoid; and medium-chain-triglyceride (MCT) oil.

2. The preparation of claim 1,
wherein the cannabinoid is included in an amount of about 1 wt % to about 20 wt % relative to a total weight of the preparation.

3. The preparation of claim 1,
wherein the MCT oil is included in an amount of about 0.1 wt % to about 1 wt % relative to a total weight of the preparation.

4. The preparation of claim 1,
wherein the preparation further comprises a film-forming agent,
wherein the film-forming agent comprises maltodextrin.

5. The preparation of claim 4,
wherein the maltodextrin is included in an amount of about 10 wt % to about 40 wt % relative to a total weight of the preparation.

6. The preparation of claim 4,
wherein the film-forming agent comprises a combination of maltodextrin and pullulan.

7. The preparation of claim 1,
further comprising an excipient or an additive,
wherein the excipient is at least one selected from a plasticizer, a chelating agent, a disintegrant, a stabilizer, a colorant, or a dye, and
the additive is at least one selected from vitamins, a nutritional enhancer, an acidity regulator, minerals, a sweetener, a souring agent, a flavoring agent, a flavor enhancer, or an antifoaming agent.

8. A method of preparing the oral disintegrating using film preparation of claim 1, the method comprising: heating and dissolving a mixture of cannabinoids and a solvent, followed by cooling;
preparing a film-forming solution by adding an MCT oil and a film-forming agent to the mixture and stirring; and
drying the film-forming solution.

9. The method of claim 8,
wherein the preparing of the film-forming solution further comprises adding an excipient or an additive, followed by stirring,
wherein the excipient is at least one selected from a plasticizer, a chelating agent, a disintegrant, a stabilizer, a colorant, or a dye, and
the additive is at least one selected from vitamins, a nutritional enhancer, an acidity regulator, minerals, a sweetener, a souring agent, a flavoring agent, a flavor enhancer, or an antifoaming agent.

10. The method of claim 8,
wherein the drying is carried out at a temperature of about 70° C. to about 90° C.
